(19) United States
(12) Patent Application Publication
(10) Pub. No.: US 2011/0053832 A1
(43) Pub. Date: Mar. 3, 2011

(54) NATURAL ANTIMICROBIAL COMPOSITION

(75) Inventors: Marie N. ANTONIEWSKI, Chicago, IL (US); Charles A. Kennett, Brookfield, IL (US); Meghan Anne Mcilroy, Park Ridge, IL (US); Zuoxing Zheng, Palatine, IL (US); Judith Gulten Moca, Palatine, IL (US); Sandra E. Kelly-Harris, Hazel Crest, IL (US)

(73) Assignee: KRAFT FOODS GLOBAL BRANDS LLC, Northfield, IL (US)

(21) Appl. No.: 12/553,438

(22) Filed: Sep. 3, 2009

(51) Int. Cl. A61K 38/12

(52) U.S. Cl. 514/2.3; 514/2.9

(57) ABSTRACT

Natural antimicrobials for foods, such as salad dressings and dairy products to target a broad spectrum of food emulsion spoilage microorganisms such as lactic acid bacteria and fungi such as general yeast and even acid-tolerant yeast (such as Zygosaccharomyces bailii). The formulations include at least a combination of a nisin or a nisin containing ingredient and an organic acid and/or its salt. Formulations can include cultured antibacterials that produce nisin and can be combined with organic acids/salts of, for example, acetic acid and Na-Acetate or Ca-Acetate. One formulation where broad spectrum spoilage inhibition is achieved includes nisin or a nisin containing cultured ingredient and 3 percent Ca-Acetate.

### FIELD

The present compositions relate generally to natural antimicrobials and specifically to natural antimicrobials using organic acids and their salts in combination with cultured ingredients to inhibit growth of spoilage organisms in food products including emulsions, dairy products and the like.

### BACKGROUND

Food products are susceptible to spoilage by a variety of microbial agents. Reducing food spoilage and increasing shelf life of processed foods in the past has included various combinations of heat, pressure, irradiation, ultrasound, refrigeration, natural and artificial antimicrobial/preservative compositions, and the like. Any useful antimicrobial process or composition should target food specific spoilage agents and minimize its affect on the food products themselves. Antimicrobial compositions have been effective in reducing food spoilage and extending shelf life of food emulsions and dairy products, such as water continuous emulsions of salad dressing, mayonnaise, and the like, and dairy products such as cottage cheese and cream cheese.

Although many antimicrobial compositions are made with artificial components, there are also known attempts to use cultured ingredients. These compositions can include cultured ingredients containing a lantibiotic such as nisin. Lantibiotics are bacteriocins generally produced by a Gram-positive bacterium to attack other Gram-positive bacteria. Thus, a bacteriocin is a toxin produced by the bacteria to inhibit the growth of similar or closely related bacterial strains of lactic acid bacteria that can cause food spoilage.

Nisin can be produced by culturing of the bacterium Lactococcus lactis or Streptococcus lactis on natural substrates, such as milk or dextrose. Additionally, nisin can also be produced by recombinant technology. Nisin is effective against many Gram-positive organisms, including lactic acid bacteria (commonly associated with spoilage of processed cheese, milk or cream) and Listeria monocytogenes (a known pathogen). Nevertheless, nisin and nisin containing ingredients are not known for antifungal activity against yeasts and molds.

Many other types of antimicrobial compositions include a plurality of antimicrobial agents to address spoilage organisms of specific food types including combining, among other things, the bacteriocins from cultures of the proprionibacteria genus of lactic acid bacteria with organic acids and their salts. (See JP 07-115950). U.S. Pat. No. 5,217,950 to Blackburn et al. discloses a broad range bactericide that is a combination of nisin with a chelating agent such as EDTA or other acetate salt or citrate salt. Antibacterial compositions that include propionibacterial metabolites in combination with two or more of the following: a lantibiotic; a lytic enzyme; and an organic acid or its salt are also known.

Some compositions are considered effective against Gram-positive and Gram-negative vegetative bacteria plus harmful Gram-positive spore forming bacteria. The compositions can include incorporating viable propionibacteria directly into a food, along with an organic acid or its salt. Alternatively, the propionibacterium cultures may contain skim milk or other fermentation medium from which propionibacterial metabolites can be purified then added to a food product. The target food product may include water continuous emulsions such as salad dressings and soups.

Further, U.S. Pat. No. 3,899,594 (hereinafter “Nickerson”) discloses adding an inoculum Pediococcus cerevisiae or Lactobacillus viridescens and a combination of a sorbic acid salt and a propionic acid salt to a food product. The inoculum grows in the presence of the sorbic acid and propionic acid salts, which inhibit some types of undesirable food bacteria.

While there have been significant advances in the art, further advances are possible and desired. Recent consumer demand has placed an increased emphasis on providing foods that are more natural and fresh, and with less processing. Thus, it would be desirable to provide more natural antimicrobial compositions to inhibit a broad range of spoilage organisms in food products. A specific need in the art is a more natural antimicrobial for food emulsions, such as creamy salad dressings.

### SUMMARY

Accordingly, there is provided herein natural antimicrobial compositions for shelf stable water continuous emulsions, such as salad dressings to target a broad spectrum of food emulsion spoilage microorganisms such as lactic acid bacteria and fungi such as general yeast and even acid-tolerant yeast (such as Zygosaccharomyces bailii). The formulations include at least a combination of a cultured antimicrobial and an organic acid and/or its salt. The cultured antimicrobial can be nisin or a nisin-containing ingredient and can be combined with organic salts of, for example, sodium acetate or calcium acetate.

In one embodiment, the natural antimicrobial for foods has a nisin component in the range of 2-40 ppm nisin; and an undissociated organic acid/salt. An undissociated ion of acetic acid/acetate can be greater than or equal to 0.5% formula. The undissociated organic acid/salt ion can be lactic/lactate, citric/citrate or propionic/propionate, and can be greater than or equal to about 0.5% formula.

In a specific embodiment, an antimicrobial can have 3 percent of total weight Na-Acetate with an undissociated organic acid/salt ion of at least 0.5% and the nisin component can be derived from a percent total weight liquid cultured milk in the range of about 6 to 12 percent or from about a 2 to 4 percent total weight powdered cultured sugar.

Other embodiments can include an undissociated organic acid/salt of about 3 percent of total weight Ca-Acetate with an undissociated organic acid/salt ion of at least 0.5% and the nisin component can be derived from a 6 percent total weight liquid cultured milk acidified with glacial acetic acid.

In another specific embodiment the undissociated organic acid/salt can be a percent of total weight Ca-Acetate.

Other aspects of the antimicrobial can include a pH in the range of about 2 to 6.

Other features will become more apparent to persons having ordinary skill in the art to which the antimicrobial pertains and from the following description and claims.

### BRIEF DESCRIPTION OF THE FIGURES

The foregoing features, as well as other features, will become apparent with reference to the description and figures below, in which like numerals represent elements, and in which:

FIG. 1 illustrates a comparison of 1.5 percent Ca-Acetate and different levels of cultured milk and nisin alone and in combination for inhibition of generic yeast.

FIG. 2 illustrates a comparison of 3 percent Ca-Acetate and different levels of cultured milk alone and in combination for inhibition of Z. bailii

### DETAILED DESCRIPTION

Described herein are antimicrobial formulations, and specifically natural antimicrobials for shelf stable water continuous emulsions, such as salad dressings, soups, sauces, mayonnaise, processed cheese, cottage cheese, cream cheese, macaroni and cheese, and the like. The embodiments can be configured to target a broad spectrum of food emulsion spoilage microorganisms such as lactic acid bacteria and fungi such as general yeast and even acid-tolerant yeast (such as Zygosaccharomyces bailii). As described below, many formulations of these natural antimicrobial for emulsions are possible, but generally include at least a combination of a nisin or a nisin-containing ingredient and an organic acid and/or its salt. A nisin containing ingredient can include a cultured antimicrobial, such as a milk or sugar culture. When one of these formulations is added to a food product, it enhances its shelf life and prevents a broad spectrum of microbial growth not known in the art for natural antimicrobials. The formulations show a broad spectrum inhibition unexpected from the use of or an organic acid and/or its organic salt separately.

In short, the antimicrobial inhibition of the present formulations extends beyond the additive inhibition of its parts. As described below, the individual organic acids and/or individual organic salts, as tested, only slightly inhibited the tested food emulsion spoilage microorganisms. The cultured ingredients, as tested, also had a minimum antimicrobial effect. However, when the nisin or nisin-containing cultured ingredients were combined with certain organic acids and their salts and applied to the emulsified food product, the combination shows inhibition of the spoilage organisms and an increased inhibition against yeasts. This is remarkable given that only a few preservatives, such as Sorbic acid/Sorbate and natamycin are known to inhibit Z. bailii. These results were also remarkable given that nisin and nisin-containing cultured ingredients have not typically demonstrated effectiveness against yeast. For example, nisin and nisin-containing ingredients have been reported to have antibacterial activity, but are not known for antifungal activity. Nevertheless, the present formulations established both antibacterial and antifungal activity when combined with organic acids and their salts.

Turning now to the table, provided herein are formulations of a natural antimicrobial tested using a water continuous emulsion with a creamy texture and flavor, such as a creamy style salad dressing. It is noted that not only other types of water continuous emulsions, but also many other types of foods would also benefit from the antimicrobial nature of these formulations. As shown, the emulsion is standardized for a ratio of acid-to-moisture and salt-to-moisture values of a minimum of about 1.5 and about 4.8, respectively. When the emulsion was inoculated individually with cocktails of lactic acid bacteria, general yeast, or acid-tolerant yeast (such as Z. bailii) at approximately 4.0 logs, the emulsion spoils with microbial growth up to 7.0 logs. The overall pH of the present formulations can be anywhere from about 2.0 to about 6.0. Lower pH can often inhibit microbial growth, irrespective of other antimicrobial agents present. In the present formulations however, it was observed that the presence of antimicrobial agents such as cultured milks and/or organic acid salts were effective antimicrobial inhibitors, even at higher pH levels. High concentrations of undissociated organic acids can produce greater microbial inhibition. Increasing pH is associated with decreasing concentrations of undissociated acid. Thus, the microbial inhibition in a food that approaches a pH of 6.0 is unexpected.

As tested, antimicrobial effectiveness was presumed to be demonstrated when there was at least 1 Log negative difference between treatment and inoculated positive control without treatment and/or less than 1 Log Growth as compared to the initial micro levels of treatment. A failed treatment did not show any difference as compared to the positive control without treatment and/or had more than 1 Log Growth as compared to the initial micro levels of that treatment. The label LDPC indicates the Log Difference in micro levels as compared to the Positive Control without treatment (negative values represent degree of inhibition). The label LDIC indicates the Log Difference in micro levels as compared to the Initial Count or micro level of that treatment (negative values represent a degree of inhibition).

As described below, the present formulations can use a cultured ingredient whereby the energy source is milk, sugar, dextrose, corn syrup, or the like. Generally, the cultured ingredients contemplated for the present formulations include antimicrobial peptides (bacteriocins) and other ingredients with that mode of action, preferably lantibiotics, and more preferably nisin and nisin-containing ingredients. One possible milk culture can include raw materials (milk, whey or other dairy powder, or dextrose, corn syrup or other carbohydrates supplemented with other nutrients for bacterial growth, with or without an acid neutralizer such as calcium carbonate) inoculated with a nisin (or other bacteriocin)-producing bacteria such as Lactococcus lactis. The materials are then fermented between about 25-35 degrees C. at a pH between about 4.5 and 7 (or even without pH control) for 18-72 hours under anaerobic conditions with mild agitation.

The nisin-containing fermentates are then directly used in foods or further separated or concentrated and/or dried before direct nisin use in foods. Any form of nisin-containing fermentates, with or without further processes, including liquid and dry versions, is considered as a nisin-containing cultured ingredient. Nisin and nisin-containing components can be in the range of 2-40 ppm, preferably 5-30 ppm, and more preferably 10-20 ppm in terms of pure nisin content. As an example, if a culture contains 2.5% nisin, then its use can be in the range of about 78-1560 ppm. All nisin-containing ingredients for purposes of the formulations described herein have a minimum nisin level of 1000 IU/g as measured by determining nisin activity by the method described in U.S. patents (U.S. Pat. Nos. 6,136,351; 6,113,954; 6,613,364; 6,242,017 and 6,797,308).

It is noted that the formulations described herein liquid cultured milk is used. However, powdered culture milk formulations can also be provided and determined. For example, antimicrobial activity in 6 percent liquid cultured milk is approximately equivalent to 1.8 percent cultured milk powder. In the present formulations, the moisture in cultured milk powder is approximately 3 percent and about 80 percent in liquid cultured milk. An affective range for powdered cultured milk can be from about 0.1 percent to about 10 percent and liquid cultured milk can be from about 0.3 percent to about 30 percent. Preferably the range is from about 1 to 5 percent for powdered cultured milk and from about 3 to 15 percent for liquid cultured milk. More preferably, the range is from about 2 to 4 percent cultured milk powder and from about 6 to 12 percent liquid cultured milk.

Also, an important component of the present formulations is the use of organic acids and their undissociated salts. Specifically, the formulations can include Na-Acetate and Ca-Acetate. It is noted that the formulations can also include many undissociated edible organic acids and their salts. These can include, but are not limited to the acid/salts of acetic/acetate, propionic/propionate, lactic/lactate, and citric/citrate.

The antimicrobial results of various formulations are also shown on the table. Experiment 1 included sodium (Na) acetate (a sodium salt of acetic acid) at 3 percent total weight although the range of Na-Acetate can be from about 1.5 to about 3.0 total percent weight. In any event, the undissociated ion usage level as determined using an organic acid/salt, such as pKₐ, should be greater than or equal to 0.5 percent, preferably 0.6 percent and more preferably 0.7 percent. This formula showed significant antimycotic inhibition of Z. bailii. However, the 3 percent Na-Acetate formula did not inhibit general yeast nor lactic acid bacteria. The formula can include acetic acid of less than about 6 percent, preferably less than about 4 percent, and more preferably less than about 2 percent. For example, the formula can include about 0.4 percent total acetic acid from various combinations of vinegar and garlic juice.

Experiment 2 included a 6 percent liquid cultured milk formula that alone showed no antimicrobial effectiveness of interest for water continuous food emulsions. Specifically, it failed to inhibit lactic acid bacteria, general yeast, and Z. bailii. Three percent Na-Acetate with no added phosphoric acid inhibited all of the tested spoilage organisms. For this example, more vinegar was used to maximize the level of acetic acid. However, when 6 percent cultured milk and 3 percent Na-Acetate were combined, the ingredients showed increased efficacy of broad spectrum spoilage inhibition of lactic acid bacteria and general yeast and Z. bailii compared to the 3 percent Na-Acetate alone. It was surprising to observe that the combination of 6 percent cultured milk and 3 percent Na-Acetate had a more substantial decline in yeast population including the acid tolerant yeast than sodium acetate alone.

Experiment 3 included a 6 percent liquid cultured milk formula acidified with food-grade glacial acetic acid (GM). The added acid was to reduce the pH of the cultured milk so that its addition to a food emulsion, such as a dressing, would not unacceptably increase the overall pH of the food product. The titratable acidity of the resultant acidified cultured milk can be about 6.15 to achieve this result. This formulation inhibited lactic acid bacteria, but it failed to inhibit general yeast and acid-tolerant yeast. However, when in combination with 3 percent calcium (Ca) acetate (the calcium salt of acetic acid) improved broad spectrum inhibition was observed. The three percent Ca-Acetate, as shown inhibited the growth of lactic acid bacteria and general yeast but not Z. bailii. It is noted that the range of Ca-Acetate can be in the range of about 1.5 to 3.0 total percent weight. In any event, the undissociated ion usage level should be greater than or equal to 0.5 percent, preferably 0.6 percent and more preferably 0.7 percent. When combined with 6 percent cultured milk, increased broad spectrum inhibition of spoilage organisms is found including significant antimicrobial efficacy against Z. bailli. Similarly, 2 percent cultured sugar significantly inhibited lactic acid bacteria, but showed no mycotic inhibition of general yeast or Z. bailii (not shown). When 2 percent cultured sugar was combined with 3 percent Na-Acetate, the ingredients showed increased broad spectrum inhibition of lactic acid bacteria, general yeast and Z. bailii.

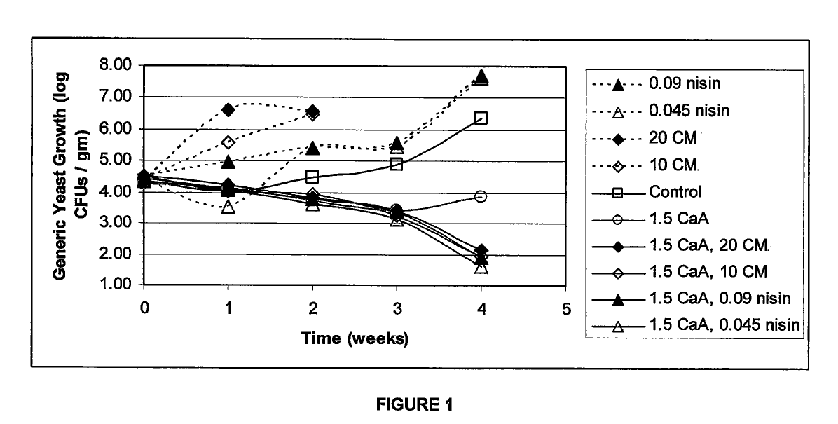

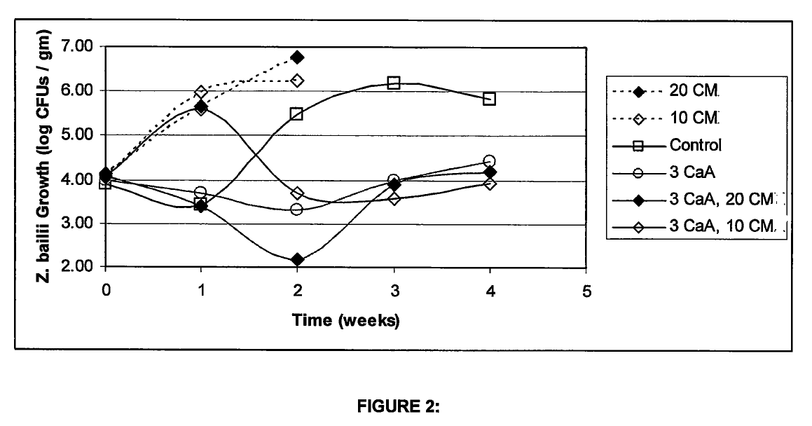

Experiment 4 and FIGS. 1 and 2 demonstrate increased antimicrobial inhibition and sometimes synergistic antimicrobial inhibition when using various combinations of Ca-Acetate, nisin, and/or nisin-containing cultured ingredients added to inoculation studies of a creamy salad dressing. Two levels of Ca-Acetate alone and in combination with two levels of nisin and a nisin containing cultured ingredient were compared to determine their effect on typical spoilage (generic) yeast, the preservative resistant yeast, Z. bailii, and Lactobacillus.

Specifically, the samples were subjected to three different inocula (at log CFU 3-4) that contained microorganisms previously shown to spoil salad dressing. These included a cocktail of seven strains comprising various genera of yeast not known to be preservative resistant, a three strain cocktail of the preservative resistant Zygosaccharomyces bailii, and a three strain cocktail of Lactobacillus spp. (L. plantarum, L. fermentum, L. buchneri). The sample was then incubated at 22 degrees Celsius, and tested weekly for four weeks for the presence of these microorganisms.

FIG. 1 illustrates a comparison of 1.5 percent Ca-Acetate and different levels of nisin-containing cultured ingredient and nisin alone and in combination for inhibition of generic yeast. FIG. 2 illustrates a comparison of 3 percent Ca-Acetate and different levels of nisin containing cultured ingredient alone and in combination for inhibition of Z. bailii.

Generally as shown, 3 percent Ca-Acetate alone is needed to inhibit Z. bailii; 1.5 percent Ca-Acetate is needed to inhibit generic yeast; nisin or a nisin containing cultured ingredient are needed to inhibit Lactobacillus; nisin or a nisin containing cultured ingredient with Ca-Acetate work to synergistically inhibit general yeast; the nisin containing cultured ingredient may increase inhibitory effect of Ca-Acetate against Z. bailii; and broad spectrum spoilage inhibition is achieved with a combination of 3 percent Ca-Acetate and either a nisin containing cultured ingredient or nisin alone.

While natural antimicrobials for shelf stable water continuous emulsions have been described in conjunction with specific embodiments, it is evident that many alternatives, modifications, and variations will be apparent to those skilled in the art in light of the foregoing description.

TABLE

| | | Salt of | | | | | Treatment Performance | | | | | | | | |
|---|---|---|---|---|---|---|---|---|---|---|---|---|---|---|---|
| | | Organic | Cultured | | | | Generic Yeast | | | Lactobacillus | | | Z. bailii | | |
| Exp | Formula | Acid (%) | Ingredient (%) | A/M | S/M | pH | Pass | LDPC | LDIC | Pass | LDPC | LDIC | Pass | LDPC | LDIC |
| 1 | Control | 0 | 0 | 1.38 | 5.18 | 3.53 | | 0 | 2 | | 0 | −3 | | 0 | 3 |
| | 3% Na Ac | 0 | 3 | 1.4 | 5.16 | 5.2 | | −0.5 | 1 | | 0 | −3.5 | [symbol] | −3 | −1 |
| 2 | Control | 0 | 0 | 1.54 | 5.56 | 3.72 | | 0 | −3.5 | | 0 | −3.5 | | 0 | 3 |
| | 3% Na acetate | 0 | 3 | 1.59 | 5.51 | 5.14 | [symbol] | 0 | −3.5 | [symbol] | 0 | −3.5 | [symbol] | −2 | −0.5 |
| | 6% CM (liquid) | 6 | 0 | 1.50 | 5.31 | 4.00 | | 3 | −0.5 | | 3 | −0.5 | | 0 | 1.5 |
| | 3% Na acetate + | 6 | 3 | 1.52 | 5.42 | 5.31 | [symbol] | 0 | −3.5 | [symbol] | 0 | −3.5 | [symbol] | −2.5 | −1.5 |
| | 6% CM (liquid) | | | | | | | | | | | | | | |
| 3 | Control | 0 | 0 | 1.55 | 5.32 | 3.46 | | 0 | 2.5 | | 0 | 0 | | 0 | 1 |
| | 6% CM (liquid) | 6 | 0 | 1.56 | 5.19 | 4.00 | | −0.5 | 2.5 | [symbol] | −2.4 | −2 | | 0 | 1.5 |
| | 3% Ca acetate | 0 | 3 | 1.53 | 5.24 | 4.85 | [symbol] | −3 | −1 | [symbol] | −2.5 | −2.5 | | 0.5 | 1.5 |
| | 3% Ca acetate + | 6 | 3 | 1.52 | 5.27 | 4.92 | [symbol] | −2 | 0 | [symbol] | −3.5 | −3.5 | [symbol] | −2.5 | −1.5 |
| | 6% CM (liquid) | | | | | | | | | | | | | | |
| 4 | Control | 0 | 0 | 1.56 | 5.32 | 3.39 | | 0 | 2 | | 0 | −1 | | 0 | 2 |
| | CM (mid) (liquid) | 0 | 10 | 1.52 | 5.27 | 4.15 | | 0 | 2 | [symbol] | −2.5 | −2.5 | | 0.5 | 2 |
| | CM (high) (liquid) | 0 | 20 | 1.51 | 5.22 | 4.47 | | 0 | 2 | [symbol] | −2.5 | −2.5 | | 1 | 2.5 |
| | Ca acetate (mid) | 1.5 | 0 | 1.56 | 5.22 | 4.70 | [symbol] | −2.5 | −0.5 | | −0.5 | −1.5 | | 0.5 | 2 |
| | Ca acetate (high) | 3 | 0 | 1.59 | 5.26 | 4.94 | [symbol] | −4 | −2 | | 1.5 | 0.5 | [symbol] | −1.5 | 0.5 |
| | Ca acetate (mid), | 1.5 | 10 | 1.58 | 5.25 | 4.81 | [symbol] | −4.5 | −2.5 | [symbol] | −2.5 | −2.5 | | 0.5 | 2 |
| | CM (mid) (liq) | | | | | | | | | | | | | | |
| | Ca acetate (mid), | 1.5 | 20 | 1.56 | 5.20 | 4.97 | [symbol] | −4 | −2.5 | [symbol] | −1.5 | −2.5 | | 0.5 | 2 |
| | CM (high) (liq) | | | | | | | | | | | | | | |
| | Ca acetate (high), | 3 | 10 | 1.64 | 5.33 | 5.04 | [symbol] | −2.5 | −1 | [symbol] | −2.5 | −3 | [symbol] | −2 | 0 |
| | CM (mid) (liq) | | | | | | | | | | | | | | |
| | Ca acetate (high), | 3 | 20 | 1.57 | 5.19 | 5.13 | [symbol] | −4 | −2 | [symbol] | −2.5 | −3 | [symbol] | −1.5 | 0 |
| | CM (high) (liq) | | | | | | | | | | | | | | |
| | Nisin (mid) | 0 | 1.13E−03 | 1.51 | 5.18 | 3.50 | | 1.5 | 3.5 | [symbol] | −2.5 | −1 | | 0 | 2 |
| | Nisin (high) | 0 | 2.25E−03 | 1.53 | 5.19 | 3.17 | | 1.5 | 3 | [symbol] | −2.5 | 0 | | 0 | 2 |
| | Ca acetate (mid), | 1.5 | 1.13E−03 | 1.55 | 5.30 | 4.73 | [symbol] | −5 | −2.5 | [symbol] | −2.5 | −3 | | −0.5 | 1.5 |
| | Nisin (mid) | | | | | | | | | | | | | | |
| | Ca acetate (mid), | 1.5 | 2.25E−03 | 1.60 | 5.21 | 4.72 | [symbol] | −4.5 | −2.5 | [symbol] | −2.5 | −2 | | 0 | 2 |
| | Nisin (high) | | | | | | | | | | | | | | |
| | Ca acetate (high), | 3 | 1.13E−03 | 1.64 | 5.27 | 4.90 | [symbol] | −4 | −2 | [symbol] | −2.5 | −2.5 | [symbol] | −1.5 | 0.5 |
| | Nisin (mid) | | | | | | | | | | | | | | |
| | Ca aceate (high), | 3 | 2.25E−03 | 1.60 | 5.25 | 4.95 | [symbol] | −4 | −2 | [symbol] | −2.5 | −2.5 | [symbol] | −1.5 | 0.5 |
| | Nisin (high) | | | | | | | | | | | | | | |

1. A natural antimicrobial for foods, comprising:
a nisin component in the range of 2-40 ppm nisin; and
an undissociated organic acid/salt.

2. The antimicrobial of claim 1, wherein an undissociated ion of acetic acid/acetate is greater than or equal to 0.5% formula.

3. The antimicrobial of claim 1, wherein the undissociated organic acid/salt is lactic/lactate, and is greater than or equal to 0.5% formula.

4. The antimicrobial of claim 1, wherein the undissociated organic acid/salt is citric/citrate, and is greater than or equal to 0.5% formula.

5. The antimicrobial of claim 1, wherein the undissociated organic acid/salt is propionic/propionate, and is greater than or equal to 0.5% formula.

6. The antimicrobial of claim 1, wherein the undissociated organic acid/salt is at least 3 percent of total weight Na-Acetate with an undissociated organic acid/salt ion of at least 0.5% and the nisin component is derived from percent total weight liquid cultured milk in the range of about 6 to 12 percent.

7. The antimicrobial of claim 1, wherein the undissociated organic acid/salt is at least 3 percent of total weight Na-Acetate with an undissociated organic acid/salt ion of at least 0.5% and the nisin component is derived from about 2 to 4 percent total weight powdered cultured sugar.

8. The antimicrobial of claim 1, wherein the undissociated organic acid/salt is at least 3 percent of total weight Ca-Acetate with an undissociated organic acid/salt ion of at least 0.5% and the nisin component is derived from an about 6 percent total weight liquid cultured milk acidified with glacial acetic acid.

9. The antimicrobial of claim 1, wherein the undissociated organic acid/salt is about 3 percent of total weight Ca-Acetate with an undissociated organic acid/salt ion of at least 0.5%.

10. The antimicrobial of claim 1, wherein the pH of the antimicrobial is in the range of about 2 to about 6.

11. The antimicrobial of claim 6, wherein the percent total weight liquid cultured milk is 6 percent.
